(19) United States
(12) Patent Application Publication
(10) Pub. No.: US 2016/0271363 A1
(43) Pub. Date: Sep. 22, 2016

(54) STIFFNESS ADJUSTABLE CATHETER

(71) Applicant: ABBOTT CARDIOVASCULAR SYSTEMS INC., Santa Clara, CA (US)

(72) Inventors: Jochen Bauer, Rielasingen (DE); Aaron M. Fortson, Freemont, CA (US); John M. Ziobro, Centerville, MN (US); David J. Milazzo, Santa Clara, CA (US)

(73) Assignee: ABBOTT CARDIOVASCULAR SYSTEMS INC., Santa Clara, CA (US)

(21) Appl. No.: 14/442,259

(22) PCT Filed: Nov. 4, 2013
(86) PCT No.: PCT/US2013/068314

§ 371 Date: May 12, 2015

Related U.S. Application Data
(60) Provisional application No. 61/781,349, filed on Mar. 14, 2013.

(51) Int. Cl. A61M 25/00

(52) U.S. Cl. CPC A61M 25/0054; A61M 25/0026; A61M 25/0043; A61M 2025/0059; A61M 2210/12; A61M 2025/0063; A61M 25/0108

(57) ABSTRACT

Adjustable catheter including an elongate tubular shaft having a proximal portion, a distal portion, and a length therebetween. The elongate tubular shaft has a guidewire lumen defined therein extending along at least the distal portion of elongate tubular member and further has at least one pressure lumen defined therein extending along at least a section of the length of the elongate tubular shaft. The at least one pressure lumen having a proximal end and a closed distal end. A fluid adaptor is in fluid communication with the at least one pressure lumen. The catheter has a stiffness profile and a flexibility profile along a length there of and at least one of the stiffness profile and the flexibility profile selectively adjustable upon introduction of a pressurizing fluid into the at least one pressure lumen.

### CROSS-REFERENCE TO RELATED APPLICATION

This application claims the benefit of U.S. Provisional Patent Application Ser. No. 61/781,349, entitled “Stiffness Adjustable Catheter” and filed on Mar. 14, 2013, the entire content of which is incorporated herein by reference.

### BACKGROUND OF THE DISCLOSED SUBJECT MATTER

1. Field of the Disclosed Subject Matter

The disclosed subject matter relates to stiffness adjustable catheters for treating the luminal systems of a patient. Specifically, the disclosed subject matter relates to catheters having a stiffness profile and/or a flexibility profile selectively adjustable by introduction of a pressurizing fluid.

2. Description of the Related Art

A variety of catheter devices are known in the art for treating the luminal system of a patient. Of such devices, many are directed to treating vascular systems, including the cardiovascular system and/or the peripheral system of a patient. For example, the treatment of the cardiovascular system can include the performance of angioplasty or delivery of balloon-expandable or self-expanding interventional devices (e.g., stents, filters, coils). The treatment of the peripheral system includes treatment of the carotid, popliteal and renal vessels.

One such cardiovascular system treatment includes percutaneous transluminal coronary angioplasty (PTCA), a procedure for treating heart disease. This procedure generally entails introducing a catheter assembly into the cardiovascular system of a patient via the brachial or femoral artery, and advancing the catheter assembly through the coronary vasculature until a balloon portion thereon is positioned across an occlusive lesion. Once in position across the lesion, the balloon is inflated to a predetermined size to radially compress against the atherosclerotic plaque of the lesion to remodel the vessel wall. Subsequently, the balloon is deflated to allow the catheter assembly to be withdrawn from the vasculature.

With treatment of the peripheral system, conventional catheters are configured to treat a specific type of lesion. For example, known catheters generally are configured by size to treat short, long, diffuse, or focal lesions as desired. However, such catheters can not be adapted for treatment of different lesions of various sizes or lengths. As such, it is necessary to select in advance the corresponding balloon catheter to treat the lesion of interest. However, conventional catheters are not configured to treat multiple lesions at a single time.

Furthermore the site of the occlusive lesion can only often only be reached by a tortuous pathway through the vasculature of the patient. The difficulty in accessing such regions requires that a successful catheter must be sufficiently flexible longitudinally to follow the tortuous path to the desired site, yet sufficiently stiff axially to allow the distal end of the catheter to be pushed or otherwise manipulated from an external access location.

To address this problem, catheters having varied flexibility and stiffness along their length have been developed. For example, each of U.S. Pat. No. 4,782,834 to Maguire and U.S. Pat. No. 5,370,655 to Burns discloses a catheter having sections along its length which are formed from materials having a different stiffness; U.S. Pat. No. 4,976,690 to Solar discloses a catheter having an intermediate waist portion which provides increased flexibility along the catheter shaft; U.S. Pat. No. 5,423,754 to Cornelius discloses a catheter having a greater flexibility at its distal portion due to both a material and dimensional transition in the shaft; and U.S. Pat. No. 5,649,909 to Cornelius discloses a catheter having a proximal portion with greater stiffness due to the application of a polymeric coating thereto, the contents all of which are incorporated herein by reference in their entireties.

Such conventional methods and systems generally have been considered satisfactory for their intended purpose. However, there remains a continued need in the art for an improved catheter having varied flexibility to enhance pushability, kink resistance and versatility.

In addition to PTA, PTCA, and atherectomy procedures, it also may be desirable to use adjustable balloon catheters for the peripheral system such as in the veins system or the like. Unlike balloons used for cardiovascular indications, however, balloons for peripheral indications or treatments are generally much longer in length, for example, approximately 220 mm or more. Such long length catheter balloons typically are of a fixed stiffness and flexibility. Catheter balloons typically are of a fixed length and diameter, necessitating the use of different sizes of balloons, for example, to treat vessels of varying diameter and lesions or occlusions of varying lengths.

In light of the foregoing, there is a need for an improved balloon catheter having enhanced pushability and crossability, adjustability of the balloon catheter in vivo, and enhanced protectability of any drugs positioned on an expandable member of the balloon catheter. Embodiments of the disclosed subject matter provide solutions for these issues.

### SUMMARY OF THE DISCLOSED SUBJECT MATTER

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims hereof, as well as from the appended drawings.

To achieve these and other advantages and in accordance with the purpose of the disclosed subject matter, as embodied and broadly described, the disclosed subject matter includes an adjustable catheter comprising an elongate tubular shaft having a proximal portion, a distal portion, and a length therebetween. The elongate tubular shaft has a guidewire lumen defined therein extending along at least the distal portion of elongate tubular member and further has at least one pressure lumen defined therein extending along at least a section of the length of the elongate tubular shaft. The at least one pressure lumen has a proximal end and a closed distal end. A fluid adaptor is in fluid communication with the at least one pressure lumen. The catheter has a stiffness profile and a flexibility profile along a length thereof wherein at least one of the stiffness profile and the flexibility profile is selectively adjustable upon introduction of a pressurizing fluid into the at least one pressure lumen.

Further in accordance with another aspect of the disclosed subject matter, a method of intralumen treatment is provided. The method includes providing an adjustable catheter including an elongate tubular shaft having a proximal portion, a distal portion, and a length therebetween. The elongate tubular shaft has a guidewire lumen defined therein extending along at least the distal portion of elongate tubular member. The elongate tubular shaft further has at least one pressure lumen defined therein extending along at least a section of the length of the elongate tubular shaft. The at least one pressure lumen has a proximal end and a closed distal end. The catheter further includes a fluid adaptor in fluid communication with the at least one pressure lumen, wherein the catheter has a stiffness profile and a flexibility profile along a length thereof. At least one of the stiffness profile and the flexibility profile is selectively adjustable upon introduction of a pressurizing fluid into the at least one pressure lumen. The method further includes introducing a pressurizing fluid through the adaptor to pressurize the at least one pressure lumen to vary at least one of the stiffness profile or the flexibility profile of the adjustable catheter.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the devices of the disclosed subject matter. Together with the description, the drawings serve to explain the principles of the disclosed subject matter.

### BRIEF DESCRIPTION OF THE DRAWINGS

FIG. 1 is a side view of a representative balloon catheter in accordance with the disclosed subject matter.

FIG. 2 is a cross sectional view of the catheter of FIG. 1 taken along line 2-2, which depicts a co-axial configuration for illustration and not limitation, according to an embodiment of the disclosed subject matter.

FIG. 3 is a cross-sectional view of an elongate tubular member having a multilumen configuration, according to another embodiment of the disclosed subject matter.

FIG. 4 depicts a schematic cross sectional side view of a representative micro catheter in accordance with the disclosed subject matter.

FIG. 5 depicts side view of an elongate tubular member having a multilumen configuration, according to another embodiment of the disclosed subject matter.

FIG. 6 depicts a graph of stiffness along a length of a catheter, according to another embodiment of the disclosed subject matter.

### DETAILED DESCRIPTION

Reference will now be made in detail to embodiments of the disclosed subject matter, an example of which is illustrated in the accompanying drawings. The method of the disclosed subject matter will be described in conjunction with the detailed description of the system.

As disclosed herein, the systems and methods presented herein can be used for treating the luminal system of a patient. The disclosed subject matter is particularly suited for treatment of the cardiovascular system and the peripheral system of a patient. The treatment of the cardiovascular system can include the performance of angioplasty or delivery of balloon-expandable or self-expanding interventional devices (e.g., stents, filters, coils). The treatment of the peripheral system includes, but is not limited to, treatment of the carotid, popliteal and renal vessels. Accordingly, the present disclosed subject matter is suitable for a variety of particular endovascular vessels, as well as other luminal systems of the body.

With treatment of the peripheral system, catheters according to embodiments of the disclosed subject matter can further be used in vessels with multiple lesions, such as, but not limited to, below the knee vessels. Thus, the adjustable catheter according to an embodiment of the disclosed subject matter is not limited to long, short, diffuse, or focal lesions. The adjustable catheter can treat any combination lesions due to the ability of the catheter to adapt to specific lesions.

In accordance with one aspect of the disclosed subject matter, an adjustable catheter is provided comprising an elongate tubular shaft having a proximal portion, a distal portion, and a length therebetween. The elongate tubular shaft has a guidewire lumen defined therein extending along at least the distal portion of elongate tubular member and further has at least one pressure lumen defined therein extending along at least a section of the length of the elongate tubular shaft. The at least one pressure lumen has a proximal end and a closed distal end. A fluid adaptor provided in fluid communication with the at least one pressure lumen. The catheter has a stiffness profile and a flexibility profile along a length thereof, wherein at least one of the stiffness profile and the flexibility profile selectively adjustable upon introduction of a pressurizing fluid into the at least one pressure lumen.

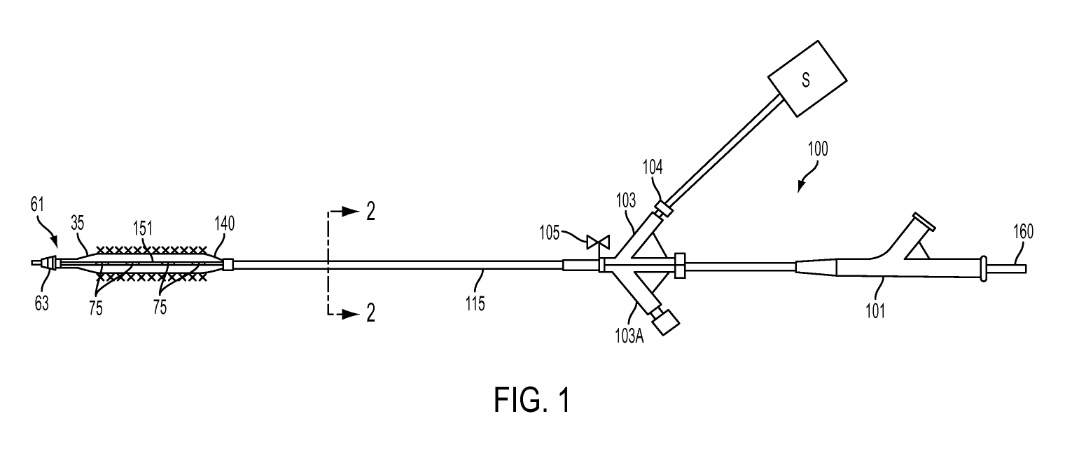

For purpose of explanation and illustration, and not limitation, a representative embodiment of an adjustable balloon, at least a portion of which is delivered within a vasculature, is shown schematically in FIG. 1. Particularly and as illustrated, the adjustable balloon catheter 100 includes an inner tubular member (or catheter shaft) 115 having a proximal end portion, a distal end portion, and a length therebetween. The inner tubular member or shaft 115 can include a variety of suitable configurations. For example, but not limitation, in the embodiment of FIG. 1, the inner tubular member comprises an over-the-wire (OTW) configuration. In this embodiment, the inner tubular member includes a guidewire tube 151 defining a guidewire lumen extending generally across the entire length of the elongate tubular shaft 115. A guidewire 160 can be introduced into the guidewire lumen 150, in a conventional manner as known.

Alternatively, the catheter can be configured with a rapid exchange configuration (RX). In this embodiment, a guidewire lumen 150 extends to a proximal guidewire port spaced distally from a proximal end portion of the inner tubular member. In either the OTW or the RX configuration, the inner tubular member can be provided with a co-axial arrangement or a multi-lumen arrangement. Further, the inner tubular member can be a single tube or an assembly of components coupled together. For purpose of example, and not limitation, the inner tubular member of the catheter embodied herein for peripheral vascular use includes a tapered L12 shaft with an outer diameter of approximately 1.08 mm, and a length of approximately 120 cm. Those skilled in the art will recognize that other configurations and known materials of construction can be used without departing from the scope of the disclosed subject matter.

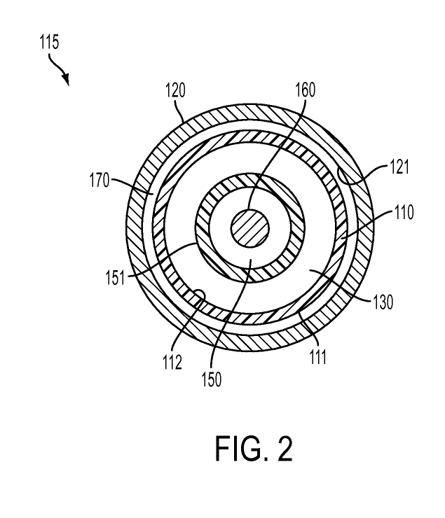

With reference to the embodiment of FIG. 2, for the purpose of illustration and not limitation, a representative cross-sectional view of a co-axial arrangement is provided. The cross-sectional view is taken along lines 2-2 of FIG. 1. As depicted, the elongate tubular shaft 115 includes an outer tubular member 120 and an inner tubular member 110. A pressurizing lumen 170 is defined annularly between the outer tubular member 120 and an exterior surface 111 of the inner tubular member 110. The inner tubular member 110 can further include a guidewire tube 151 defining a guidewire lumen 150 therein. A guidewire 160 can be introduced into the guidewire lumen 150, in a conventional manner as known. The elongate tubular shaft 115 can further include an inflation lumen 130 extending along the length of the elongate tubular shaft. In this embodiment, the inflation lumen 130 is defined annularly between an interior surface 112 of the inner tubular member 110 and the guidewire tube 151. In certain embodiments, the guidewire lumen 150 can be formed by a thin membrane of suitable strength to prevent the guidewire 160 from penetrating therethrough and minimize increases in cross-sectional profile of the inner tubular member. Alternatively, the guidewire tube 151 can be a multilayer construction, such as, but not limited to, a layer of Nylon-L25, a bonding layer such as Prim, and a layer of high-density polyethylene (HDPE). As such, and as shown in FIG. 2, at least three lumens are defined along the length of the elongate shaft—a guide wire lumen, an inflation lumen and a pressure lumen having a closed distal end.

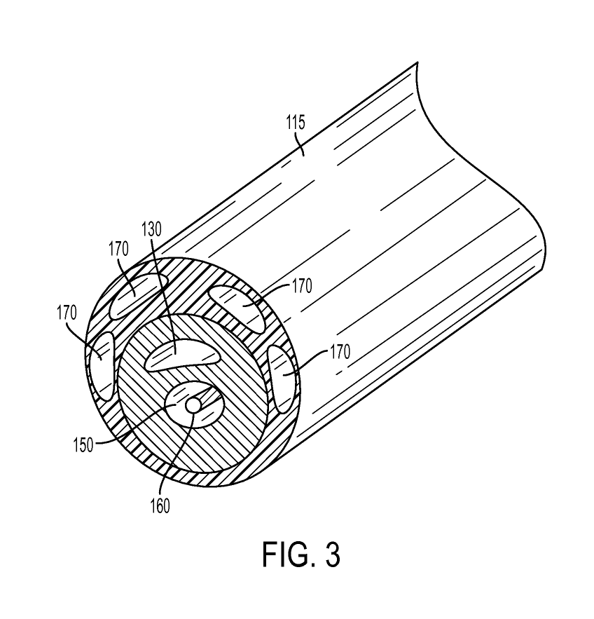

However, it is recognized that the system and method disclosed herein is not limited to a catheter having a co-axial arrangement. Rather, a monolithic, multi-lumen shaft configuration likewise can be used, wherein one or more pressure lumens are defined therein—each pressure lumen having a closed distal end. For example, and not limitation, FIG. 3 depicts a representative cross-sectional view of a multi-lumen arrangement. As depicted herein, the elongate tubular shaft 115 can be a monolithic member with the multi-lumen arrangement. In such representative embodiment, for purpose of illustration and comparison with the embodiment of FIG. 2, the elongate tubular shaft 115 defines at least one pressure lumen 170 having a closed distal end defined therein, as needed as an inflation lumen 130 and a guidewire lumen 150. The guidewire lumen 150 permits the catheter 100 to be delivered over the guidewire 160. Other suitable arrangements also can be used, wherein at least pressure lumen having a closed distal end is defined on the shaft as described herein below.

The elongate tubular shaft 115 defines the inflation lumen 130 therein. The inflation lumen is in fluid communication with an inner chamber of an expandable member 140, as described further below. The inflation lumen 130 defines a pathway for fluid to inflate the expandable member 140. Fluid can be introduced into the inflation lumen 130 at a proximal end of the catheter 100 via a luer adaptor or the like. The inflation lumen 130 can supply an inflation medium under positive pressure and can withdraw the inflation medium, e.g., by negative pressure, from the expandable member. The expandable member can thus be inflated and deflated, as further discussed below.

In an alternate embodiment, the inflation lumen and the guidewire lumen are combined and comprise a single shared lumen. For such co-axial arrangements, fluid can thus flow within the shared lumen with a guidewire positioned therein. In such configuration, the inner tubular member can comprise proximal and distal guidewire seals to sealingly engage the guidewire disposed within the fluid lumen. The shared lumen can further have a stop, alone or in addition to the seals, at the distal end to allow the guidewire to proceed past the distal end of the catheter, while preventing the fluid from escaping the shared lumen. Such co-axial configurations allow for reduced diameter of the inner tubular member, and thus reduced profile.

In either co-axial arrangement as depicted in FIG. 2 or the multi-lumen arrangement as depicted in FIG. 3, at least one pressure lumen 170 is defined within the elongate tubular shaft. In FIG. 2, the at least one pressure lumen 170 is defined between the outer tubular member 120 and the inner tubular member 110. Accordingly, the guidewire lumen 150, the at least one pressure lumen 170, and the inflation lumen 130 are in a co-axial configuration. In FIG. 3, the at least one pressure lumen 170 is defined within the elongate tubular shaft, wherein the guidewire lumen 150, the inflation lumen 130 and the at least one pressure lumen 170 extend generally parallel with each other.

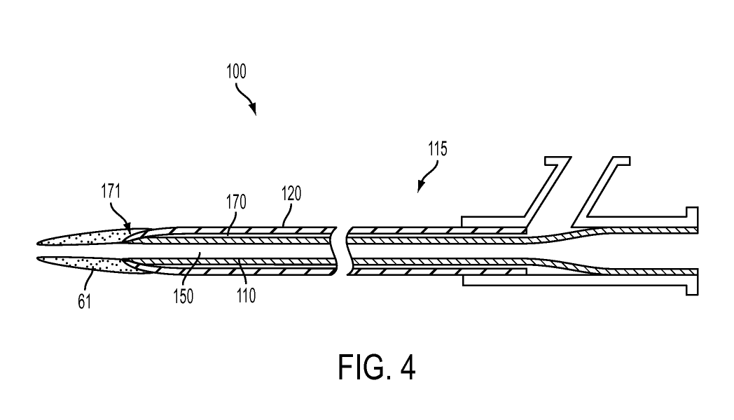

Although reference is made in the embodiments of FIGS. 1-3 to a balloon catheter having an inflation lumen defined therein, it is recognized that the system and method herein of among one or more pressure lumens, each having a closed distal end, can be used with other catheter concepts for selective flexibility and/or stiffness profiles. For example, FIG. 4 depicts a schematic cross sectional side view of a micro catheter in accordance with the disclosed subject matter. The micro catheter comprises an elongate shaft 115 with an automatic radiopaque distal tip 61. At least one pressure lumen 170 extends along at least a section of the length of the elongate tubular shaft 115 from a proximal end to a closed distal end 171. The pressure lumen 170 defines a closed pathway to contain pressurizing fluid along a selected length of the elongate tubular member 115. Pressurizing fluid can be introduced into the at least one pressure lumen 170 at a proximal end of the catheter 100. The pressure lumen 170 thus can be pressured by pressurizing fluid under positive pressure and can be voided or emptied of the pressurizing fluid, e.g., by negative pressure. As shown herein, other than a proximal entry port for the introduction/withdrawal of the pressurizing fluid, the at least one pressure lumen is free of other flow ports. The pressure lumen(s) can be pressurized using any suitable fluid medium, including but not limited to water, contrast agents, or saline solution. As embodied herein, for illustration, the micro catheter does not include an inflation lumen or expandable member.

With reference again to FIG. 3, the at least one pressure lumen can comprise a plurality of pressure lumens 170. For purposes of example, and not limitation, FIG. 3 shows a catheter having four pressure lumens 170 spaced circumferentially within the elongate tubular shaft 115. In accordance with one aspect, each of the plurality of pressure lumens can have a uniform or similar length along the catheter.

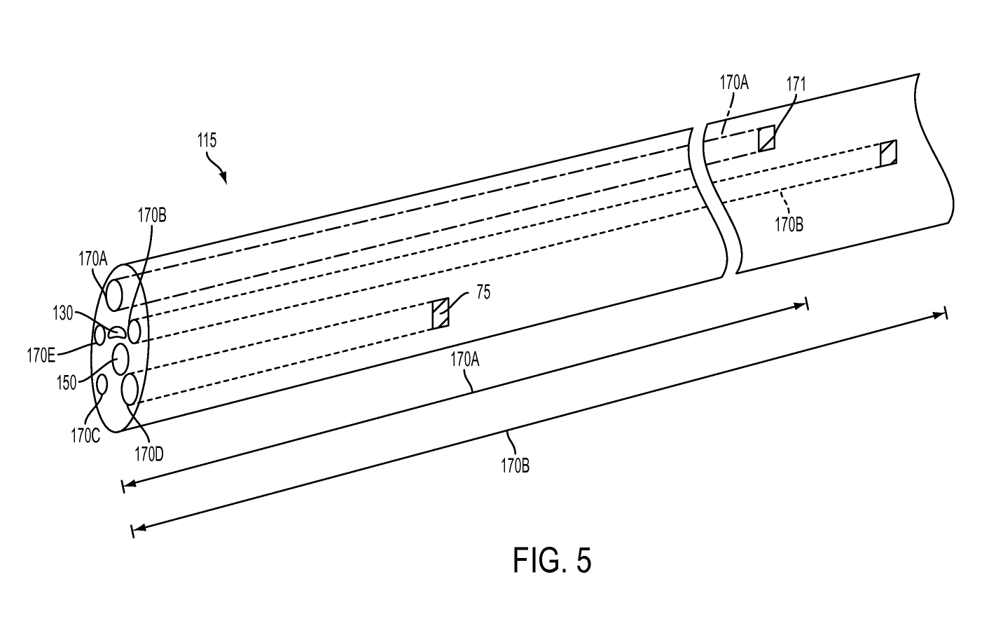

By comparison, FIG. 5 depicts another representative elongate tubular shaft 115 according to the disclosed subject matter. As depicted herein, the elongate tubular shaft 115 of FIG. 5 includes five pressure lumens 170A, 170B, 170C, 170D, and 170E. As depicted, at least one of the plurality of pressure lumens has a length different than that of the other pressure lumens. Particularly, for purpose of illustration and not limitation, each respective pressure lumen 170A-170E of the plurality of pressure lumens in the embodiment of FIG. 5 has a different length. For example, the pressure lumen 170A has a length L_{170A} and the pressure lumen 170B has a length L_{170B}. Each pressure lumen further includes at least one radiopaque marker 75 proximate the closed distal end 171 thereof. The different lengths of respective pressure lumens allows for customization of the stiffness and/or flexibility of the elongate shaft member in vivo. For example, depending on the position of the catheter along a unique tortuous path, different portions or lengths of the catheter can be provided with greater/less flexibility and stiffness as needed. By corresponding arrangement of the pressure lumens, the greater/less flexibility and stiffness of the catheter can be adjusted or otherwise provided alternatively along the desired longitudinal side of the catheter. Thus, a respective pressure lumen can be pressurized/depressurized to accommodate a wide range of issues along the tortuous paths of a intraluminal system.

As depicted in FIG. 1, a fluid adaptor 103 in fluid communication with the at least one pressure lumen 170 is depicted. The fluid adaptor 103 can be provided at the proximal end of the catheter 100 for access to the at least one pressure lumen 170 and is configured for connecting to a source S of pressurizing fluid to be coupled to the adaptor 103. For example, the fluid adaptor 103 can have multiple access ports or branches, including a luer connector at the proximal end of one branch to receive the pressurizing fluid, and a separate haemostatic valve on another branch to receive guidewire 160. Additionally, if for a balloon catheter or the like, a third branch can include a luer connector for an inflation source. A conventional device, such as but not limited to an indeflator or a syringe, can be connected to the luer connector of the pressure lumen to introduce the pressurizing fluid to the at least one pressure lumen. A locking device 104 can be provided to maintain a selected pressure of the pressurizing fluid in the at least one pressure lumen 170.

Additionally, or alternatively, another adaptor or manifold 101 can furthermore be included at the proximal end of the catheter for access to the inflation lumen 130 if provided for connecting to an inflation medium source (not shown). The manifold can have a Y-shape with a luer connector at the proximal end of one branch to receive the inflation medium, and a separate haemostatic valve on another branch to receive the guidewire 160. A conventional device, such as but not limited to an indeflator or a syringe, can be connected to the luer connector to introduce the inflation medium to the inflation lumen 130. A ratchet mechanism can further be provided to lock the operating position of the indeflator or syringe.

The indeflator or other fluid source for each of the fluid adaptor 103 or manifold 101 can be configured to control the inflation and deflation of the at least one pressure lumen 170 and inflation lumen 130, respectively. A pressure gauge can be provided with each indeflator to monitor and/or maintain the pressure system of the catheter. Each indeflator likewise can allow for the rapid release of pressure. Each indeflator can have a locking mechanism to maintain negative pressure in the catheter, which can decrease the profile of the catheter. The catheter is sized and configured for delivery within a body lumen, such as a vasculature, and particularly through a tortuous anatomy.

If a plurality of pressure lumens are provided, the catheter 100 can further include a second adaptor or branch 103A can be provided in fluid communication with a second of the plurality of pressure lumens, as depicted in FIG. 1. In this manner, the first adaptor 103 can be in fluid communication to pressure one or more of a first set of the plurality of pressure lumens, while the second adaptor can be configured to pressure one or more of a second set of the plurality of pressure lumens. Alternately or in addition thereto, the catheter can include a valve 105 in communication between the adaptor 103A and the plurality of pressure lumens to selectively pressurize at least one of the plurality of pressure lumens independent of the other pressure lumens. According to another embodiment, the plurality of pressure lumens can be in fluid communication with each other.

In accordance with another aspect of the disclosed subject matter, the catheter has a stiffness profile and a flexibility profile along a length thereof. At least one of the stiffness profile and the flexibility profile is selectively adjustable upon introduction of a pressurizing fluid into the at least one pressure lumen. Thus, the catheter 100 is customizable in vivo to allow for selectable stiffness and selectable flexibility to enhance the pushability, crossability, stiffness, and flexibility of the catheter through a tortuous anatomy.

For example, and as embodied herein, the stiffness and/or flexibility profile of the catheter can be a function of the pressurization of the at least one pressure lumen of the adjustable catheter. In addition to the pressurization of the at least one pressure lumen, a plurality of other factors can effect the stiffness of the elongate shaft member including, but not limited to, the materials of construction of the elongate shaft member, the thickness of elongate shaft member, the application of radiopaque markers, the application of a catheter tip, the length of an expandable member coupled thereto, and other applicable factors as further described. As desired, and in accordance with another aspect herein, the elongate shaft member can further include a defined section of increased axial stiffness and/or longitudinal flexibility. For instance, the tip of the catheter can provide an increased axial stiffness at the distal end of the distal end portion. The at least one pressure lumen and the plurality of factors together define the stiffness and/or flexibility profile of the elongate shaft member.

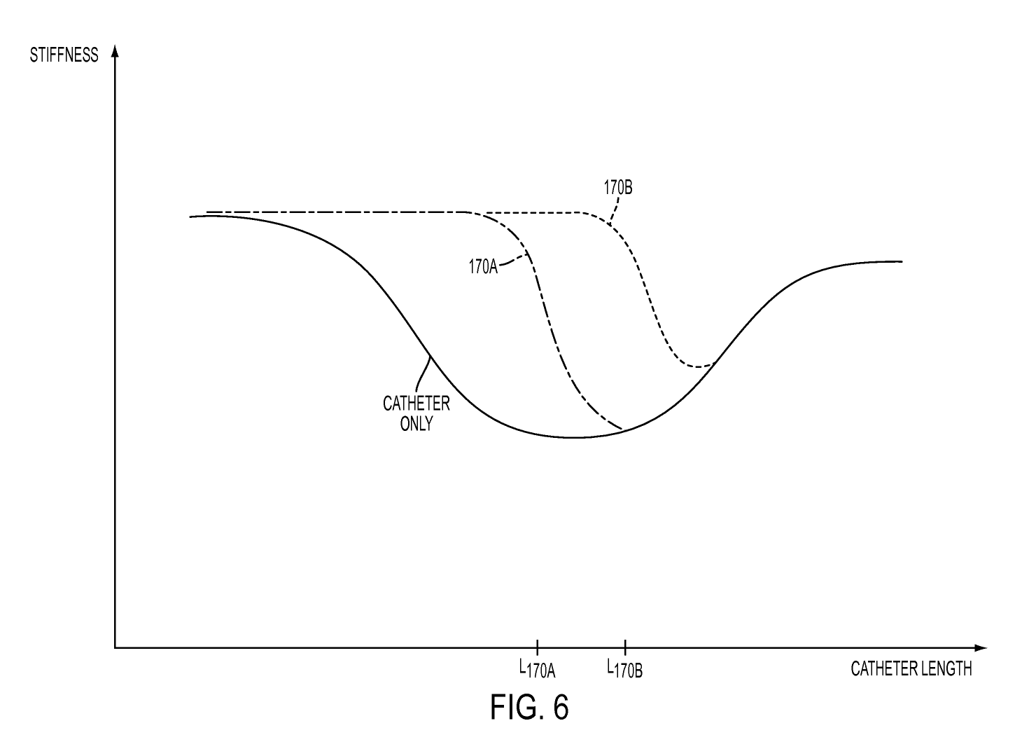

FIG. 6 depicts a graph representative of stiffness along a length of a catheter for illustration and not limitation. For example, the graph shows the stiffness profile of a catheter having the elongate tubular shaft 115 of FIG. 5. The solid line curve shows the stiffness along the catheter when none of the pressure lumens are pressurized. This solid line curve thus represents the stiffness attributed to the various factors of the catheter other than the pressurization of the at least on pressure lumen, as discussed above.

The dot-dashed line curve of FIG. 6 represents the stiffness profile of the catheter with the pressure lumen 170A of FIG. 5 pressurized. The stiffness profile of the entire catheter increases along the length of pressure lumen 170A, as depicted by the dot-dashed line curve in comparison with catheter having none of the pressure lumens pressurized, which is depicted by the solid line curve. The catheter remains more flexible immediately distal to the closed end of the pressure lumen 170A at location L_{170A} as depicted by the dot-dashed line. Distal to the location L_{170A}, the stiffness of the catheter decreases and the flexibility of the catheter increases. As depicted, the dot-dashed line curve generally coincides distally with the solid line curve at a location distal to the location L_{170A}.

The dotted line curve represents the stiffness profile of the catheter with the pressure lumen 170B of FIG. 5 pressurized. As depicted, for illustration and not limitation, the dotted line curve of 170B can follow the same initial path as the dot-dashed line curve of 170A. However, since the length of pressure lumen 170B exceeds the length of pressure lumen 170A, the catheter has greater stiffness distal to the closed end of 170A, when pressure lumen 170B is pressurized. The path of the dotted line curve deviates from the path of the dot-dashed line curve immediately distal to the closed distal end of the pressure lumen 170A. Distal to the location L_{170B}, the stiffness of the catheter decreases and the flexibility of the catheter increases. As depicted, the dotted line curve generally coincides distally with the solid line curve at a location distal to the location L_{170B}.

When more than one pressure lumen is pressurized, the resulting stiffness and flexibility profile will be a combination of the stiffness and flexibility contributed by each pressure lumen as understood by persons of ordinary skill in the art. When greater stiffness is required to pass a narrowed blood vessel, a combination of the pressure lumens can be utilized to increase the stiffness of the catheter further to allow the catheter to move distally therein.

In accordance with the disclosed subject matter and as discussed above with respect to the solid line of FIG. 6, the elongate tubular shaft can be provided with defined sections of increased flexibility and stiffness (i.e., durometer) along its length separate from the at least one pressure lumen 170. The at least one pressure lumen 170 can further supplement the defined sections of selective flexibility and stiffness. The defined sections of increased flexibility and stiffness can be accomplished in a variety of suitable ways.

In addition to the selective flexibility and stiffness of the pressure lumens, additional components can be provided to further define the desired flexibility and stiffness profile of the catheter. In one aspect of the disclosed subject matter, the elongate tubular shaft can be reinforced to provide higher stiffness of the elongate tubular shaft and thus provide a higher stiffness of the catheter itself. For example, at least a portion of the elongate tubular shaft can include a coiled construction. The coiled construction can further include a crimp at an end of the elongate tubular shaft. The crimp can further stabilize the outer diameter of the tubular member. The coiled construction can include a metallic material, such as but not limited to, stainless steel including 302, 304V, 316L; 35N LT®; CP Titanium; Pt Alloys; DFT®; Ti 6Al-4V ELI; L-605; and Nitinol. The coiled construction can further include a multi-coil construction for the inner tubular member for even greater stiffness and/or flexibility. In one embodiment, at least a portion of the coiled construction can be made of radiopaque material. In another embodiment, at least a portion of the elongate tubular shaft includes a braided construction. The braided construction can also include a metallic material, such as but not limited to, stainless steel including 302, 304V, 316L; 35N LT®; CP Titanium; Pt Alloys; DFT®; Ti 6Al-4V ELI; L-605; and Nitinol. The braided construction can have a higher density at a distal segment of the distal end portion of the inner tubular member and the higher density can function as a marker. In another embodiment, coil element or a coil tube can be disposed at the distal end thereof.

In accordance with another aspect of the disclosed subject matter, at least a portion of the elongate tubular shaft can include a hypotube. For purposes of illustration and not limitation, the hypotube can be made of suitable material, such as stainless steel or nitinol and can have a length of approximately 250 mm. For increased flexibility, the hypotube can include one or more cuts or slits, such as formed by a laser, to define flexible hinge-like regions as disclosed in U.S. Pat. Nos. 7,780,716; 7,794,489; and 7,799,065; which are incorporated by reference herein in their entirety. The hypotube can include a marker at the distal end thereof.

According to another embodiment of the disclosed subject matter, at least a portion of the elongate tubular shaft can further include a tapered shaft. The tapered shaft can increase flexibility and improve overall pushability of the catheter. According to another embodiment, the elongate tubular shaft can include a compound shaft material, and/or a member of shaft components joined together.

As previously noted, and as depicted in FIG. 1, the catheter can include an expandable member 140 coupled to the distal end portion of the elongate tubular shaft. The expandable member 140 can have a proximal end, a distal end, and a working length therebetween. The expandable member 140, or balloon as depicted herein, has an exterior surface and an interior surface. The interior surface of the expandable member defines an inner chamber 35 in fluid communication with the inflation lumen 130 of the elongate tubular shaft 115. In accordance with one aspect, the elongate tubular shaft 115 can extend at least partially through the expandable member. For purpose of illustration and not limitation, FIG. 1 shows the guidewire tube 151 extending the entire length of the balloon 140.

The expandable member 140 is transitionable between a deflated configuration and an inflated configuration. The expandable member has an overall length with a working length extending at least a portion of the overall length. The expandable member defines a longitudinal axis and can have any suitable shape along the working length thereof when in the inflated configuration. As embodied herein, for illustration and not limitation, at least a portion of the exterior surface of the expandable member along the working length is configured to engage a body lumen of a patient when the expandable member is in the inflated configuration. The expandable member can furthermore be coated with a therapeutic agent, as known in the art.

The catheter and method as disclosed herein can also be used for relatively long balloon lengths, such as peripheral balloons. For example, the expandable member can have long suitable working length of approximately 200 mm; although shorter length balloons can be used in a similar manner, such as a balloon having a working length of approximately 100 mm, for example. Additionally, the catheter can include a retractable sheath to selectively determine the working length of the balloon in vivo. Accordingly, a physician can selectively expose a desired length of the balloon to perform the desired treatment.

In embodiments with a retractable sheath disposed over the expandable member, the retractable sheath can be movable relative the elongate tubular shaft between an extended position disposed over the expandable member 140 and a retracted position proximal to the extended position. The retractable sheath is selectively positioned between the extended position and the retracted position to define an exposed length of the working length of the expandable member, as further discussed herein. By way of example, and not limitation, catheters having a retractable sheath and other features that can be used with the instant disclosed subject matter are described in International PCT Publication No. WO 2012/037510, entitled “Length and Diameter Adjustable Balloon Catheter,” and International PCT Publication No. WO 2012/037507, entitled “Length and Diameter Adjustable Balloon Catheter,” the contents all of which are incorporated herein by reference in their entirety.

As depicted in FIG. 1, the catheter can further include a stent 145 mounted at the distal end portion of the elongate tubular shaft. For example, and with reference to a balloon expandable configuration, for illustration and not limitation, the stent 145 can be positioned on an exterior surface of the balloon 140. The catheter can deliver the stent to the site of a lesion in the blood vessel. Once at the lesion, the physician can position the stent across the lesion and deploy the stent against the wall of the blood vessel (or lumen). The stent maintains its expanded configuration to maintain the patency of the blood vessel.

In accordance with another aspect of the disclosed subject matter, the elongate tubular shaft can include a distal tip configuration. For example, and as depicted in FIGS. 1 and 4, the catheter can include a distal tip 61 having a radiopaque marker 63 to enhance visibility of the distal tip within a patient's vasculature. The distal tip 61 can be coupled to the elongate tubular shaft at the distal portion thereof. The distal tip can be constructed of a soft polymer material which includes tungsten as the marker. The soft tip can prevent damage to the vessel walls while the catheter is within a patient's vasculature. The distal tip can have a lumen in communication with the guidewire lumen of the inner tubular member.

Additionally, the adjustable catheter 100 can include one or more radiopaque markers. The markers can be placed at a variety of suitable locations along the catheter. For example, and not limitation, FIG. 1 depicts markers 75 along the elongate tubular shaft within the balloon 140. Furthermore, and in accordance with the disclosed subject matter, the radiopaque markers can be strategically spaced at the closed distal end of the at least one pressure lumen, as depicted in FIG. 5. In embodiments comprising a plurality of pressure lumens, radiopaque markers can be placed at the closed distal end of each respective pressure lumen. The markers are spaced at the known distal closed end of each respective pressure lumen allowing a physician to determine which respective pressure lumen(s) to pressurize/depressurize. The markers can include any suitable material. For example, the markers can be constructed of a polymer filled or impregnated with a radiopaque material.

Further in accordance with another aspect of the disclosed subject matter, a method of intralumen treatment is provided. The method includes providing an adjustable catheter such as any of the adjustable catheter embodiments described herein. For example, the adjustable catheter can include an elongate tubular shaft having a proximal portion, a distal portion, and a length therebetween. The elongate tubular shaft has a guidewire lumen defined therein extending along at least the distal portion of elongate tubular member. The elongate tubular shaft further has at least one pressure lumen defined therein extending along at least a section of the length of the elongate tubular shaft. The at least one pressure lumen has a proximal end and a closed distal end. The catheter further includes a fluid adaptor in fluid communication with the at least one pressure lumen, wherein the catheter has a stiffness profile and a flexibility profile along a length thereof. At least one of the stiffness profile and the flexibility profile is selectively adjustable upon introduction of a pressurizing fluid into the at least one pressure lumen. The method further includes introducing a pressurizing fluid through the adaptor to pressurize the at least one pressure lumen to vary at least one of the stiffness profile or the flexibility profile of the adjustable catheter. The method further includes adjusting the pressuring fluid in the at least one pressure lumen to vary the stiffness profile or the flexibility profile of the adjustable catheter.

The catheter can include any suitable materials of construction and any suitable construction. For example, the inner and outer tubular members of the adjustable balloon catheter each can be single piece construction, or an assembly of components, and can be made of any suitable material. For example, suitable materials include, but are not limited to polymer materials such as nylon, urethane, polyurethane, PEEK, PTFE, PVDF, Kynar, PE, HDPE, a trilayer material including L25, Plexar, or polyethylene of various suitable densities. As a further exemplary alternative, the tubular members can be constructed of a composite comprising a fabrication of several different materials, such as a co-extrusion of different polymers, or a fiber-reinforced composite material such as fiber reinforced resin materials or braided materials. For example and not limitation, exemplary embodiments can include a braided tube with a PTFE liner, a Polymide middle layer with braiding and a Pebax 72D outer layer, as previously described. Furthermore, a portion of the inner and/or outer tubular shaft members can be constructed of an alloy or metallic material, such as stainless steel hypodermic tubing which is available from MicroGroup® Inc., Medway, Md. among other vendors. Other materials for the outer tubular member include PEEK; a trilayer material of L25, Plexar, HDPE; or a braided tube with a PTFE liner, a Polyimide middle layer with braiding and a Pebax 72D outer layer.

It is further contemplated that the inner and/or outer tubular shaft members can be constructed of other biocompatible material. As such, the inner and/or outer tubular shaft members of the adjustable catheter can be constructed from the above-identified polymers, combinations or blends of these polymers, whether alone or in combination with other materials, or other bioabsorbable materials.

The inner and/or outer tubular shaft members can be manufactured using a variety of known techniques such as, but not limited to, those techniques previously discussed and extrusion, injection molding, air-blowing, stretching, deep drawing, polymerization, cross-linking, dipping from solution, powder depositioning, sintering, electro-spinning, melt spinning, deformation under temperature, stretch blowing, chemical grafting any combination of the above with reinforcement element like metal braids, coils, glass fibers, carbon fibers and other kind of organic or inorganic fibers, liquid crystals, as well as classical machining technologies like milling, drilling, grinding, etc. In the event that metallic elements such as hypotubes, are to be incorporated, various metallic manufacturing techniques can be used, such as but not limited to, machining, tube drawing processes, drilling, milling EDM, other deformation methods, plating sputtering, electro grafting, sintering, and depositioning e-polishing, among others. Additionally, the inner and/or outer tubular members can be constructed from polypropylene or urethane by an extrusion process using an extruder such as that available any of a number of known suppliers, such as Medical Extrusion Technologies, Inc., Murrieta, Calif. U.S. Biosynthetic polymer materials can be constructed in a bioreactor according to the process disclosed in U.S. Pat. No. 6,495,152, the entirety of which is hereby incorporated by reference. The materials can be post processed in a number of ways including, for example and not by way of limitation, extrusion, molding, such as by injection or dipping, textile processing such as weaving or braiding, and forming. Forming processes that can be suitable are rolling and welding sheets of material or vacuum forming into tubular shapes, to name only a few examples.

The inner and/or outer tubular shaft members can be further coated with any of a variety of materials and techniques to enhance performance if desired, including a number suitable coatings and coating techniques subject to patent matters owned by Abbott Laboratories such as U.S. Pat. No. 6,541,116, U.S. Pat. No. 6,287,285, and U.S. Patent Publication No. 2002/0009535, the entireties of which are hereby incorporated by reference. For example, possible coating materials include lubricious materials such as Teflon® available from DuPont De Nemours, Wilmington, Del., U.S., and hydrophobic materials such as silicone lubricant dispersion PN 4097, available from Applied Silicone Corp., Ventura, Calif., U.S., or hydrophilic materials such as hydrogel available from Hydromer, Branchburg, N.J., U.S., or lubricious coatings such as those available from Hydro-Silk of Merritt Island, Fla., U.S.

The inner and outer tubular members can have any suitable cross-sectional shape, including elliptical, polygon, or prismatic, although a circular cross-section is common. The elongate tubular shaft can also have any suitable size and diameter depending upon the desired application. Furthermore, in the case of a balloon catheter with a “rapid exchange” (RX) guidewire design, the adjustable catheter can have an overall length between about 110 centimeters and 400 centimeters. In the case of a balloon catheter with an “over the wire” (OTW) guidewire design, the adjustable catheter can have an overall length between about 110 centimeters and 400 centimeters. In one embodiment, the adjustable catheter in accordance with the disclosed subject matter is a compatible 4 French introducer sheath BTK balloon device.

Although not illustrated, the balloon of the disclosed subject matter can have a folded non-inflated configuration with wings wrapped around the balloon to form a low profile configuration for introduction and advancement within a patient's body lumen. As a result, the balloon inflates to a nominal working diameter by unfolding and filling the molded volume of the balloon.

A wide variety of suitable materials can be used for the expandable member in accordance with the disclosed subject matter. For example, the expandable member can be made from polymeric material, including compliant, semi-compliant, or non-compliant polymeric material or polymeric blends.

In one embodiment, the polymeric material is a polyamide/polyether block copolymer (commonly referred to as PEBA or polyether-block-amide). The polyamide and polyether segments of the block copolymers can be linked through amide or ester linkages. The polyamide block can be selected from various aliphatic or aromatic polyamides known in the art. Some non-limiting examples of an aliphatic include nylon 12, nylon 11, nylon 9, nylon 6, nylon 6/12, nylon 6/11, nylon 6/9, and nylon 6/6. In one embodiment, the polyamide is nylon 12. The polyether block can be selected from various polyethers known in the art. Some non-limiting examples of polyether segments include poly(tetramethylene ether), tetramethylene ether, polyethylene glycol, polypropylene glycol, poly(pentamethylene ether) and poly(hexamethylene ether). Commercially available PEBA material can also be utilized such as for example, PEBAX® materials supplied by Arkema (France). Additionally, balloon grillamid can be used as the material for the expandable member. Various techniques for forming a balloon from polyamide/polyether block copolymer are known in the art. One such example is disclosed in U.S. Pat. No. 6,406,457 to Wang, the disclosure of which is incorporated by reference in its entirety.

In another embodiment, the expandable member is formed from polyamides. The polyamide can have substantial tensile strength, is resistant to pin-holing even after folding and unfolding, and is generally scratch resistant, such as those disclosed in U.S. Pat. No. 6,500,148 to Pinchuk, the disclosure of which is incorporated herein by reference in its entirety. Some non-limiting examples of polyamide materials suitable for the balloon include nylon 12, nylon 11, nylon 9, nylon 69 and nylon 66. Other suitable materials for constructing non-compliant balloons are polyesters such as polyethylene terephthalate (PET), Hytrel thermoplastic polyester, and polyethylene.

In another embodiment, the balloon is formed of a polyurethane material, such as TECOTHANE® (Thermedics). TECOTHANE® is a thermoplastic, aromatic, polyether polyurethane synthesized from methylene disocyanate (MDI), polytetramethylene ether glycol (PTMEG) and 1,4 butanediol chain extender. TECOTHANE® grade 1065D can be used and has a Shore durometer of 65D, an elongation at break of about 300%, and a high tensile strength at yield of about 10,000 psi. However, other suitable grades can be used, including TECOTHANE® 1075D, having a Shore D hardness of 75. Other suitable compliant polymeric materials include ENGAGE® (DuPont Dow Elastomers (an ethylene alpha-olefin polymer)) and EXACT® (Exxon Chemical), both of which are thermoplastic polymers. Other suitable compliant materials include, but are not limited to, elastomeric silicones, latexes, and urethanes.

The compliant material can be cross linked or uncrosslinked, depending upon the balloon material and characteristics required for a particular application. The polyurethane balloon materials are not crosslinked. However, other suitable materials, such as the polyolefinic polymers ENGAGE® and EXACT®, can be crosslinked. By crosslinking the balloon compliant material, the final inflated balloon size can be controlled. Conventional crosslinking techniques can be used including thermal treatment and E-beam exposure. After crosslinking, initial pressurization, expansion, and preshrinking, the balloon will thereafter expand in a controlled manner to a reproducible diameter in response to a given inflation pressure, and thereby avoid over expanding the balloon to an undesirably large diameter.

In another embodiment, the balloon is formed from a low tensile set polymer such as a silicone-polyurethane copolymer. The silicone-polyurethane can be an ether urethane and more specifically an aliphatic ether urethane such as PURSIL AL 575A and PURSIL AL10, (Polymer Technology Group), and ELAST-EON 3-70A (Elastomedics), which are silicone polyether urethane copolymers, and more specifically, aliphatic ether urethane cosiloxanes. In an alternative embodiment, the low tensile set polymer is a diene polymer. A variety of suitable diene polymers can be used such as, but not limited to, an isoprene such as an AB and ABA poly(styrene-block-isoprene), a neoprene, an AB and ABA poly(styrene-block-butadiene) such as styrene butadiene styrene (SBS) and styrene butadiene rubber (SBR), and 1,4-polybutadiene. In one embodiment, the diene polymer is an isoprene including isoprene copolymers and isoprene block copolymers such as poly(styrene-block-isoprene).

In one embodiment, the isoprene is a styrene-isoprene-styrene block copolymer, such as Kraton 1161K available from Kraton, Inc. However, a variety of suitable isoprenes can be used including HT 200 available from Apex Medical, Kraton R 310 available from Kraton, and isoprene (i.e., 2-methyl-1,3-butadiene) available from Dupont Elastomers. Neoprene grades useful in the disclosed subject matter include HT 501 available from Apex Medical, and neoprene (i.e., polychloroprene) available from Dupont Elastomers, including Neoprene G, W, T and A types available from Dupont Elastomers. Examples of other balloon and catheter embodiments which can be employed in accordance with the disclosed subject matter include U.S. Pat. Nos. 4,748,982; 5,496,346; 5,626,600; 5,300,085; and 6,406,457 and application Ser. Nos. 12/371,426; 11/539,944; and Ser. No. 12/371,422, each of which is hereby incorporated by reference in its entirety.

The expandable member can have a multilayered construction, as known in the art. Additional details and examples of suitable multilayer balloons for use in the disclosed subject matter are described in U.S. Pat. No. 7,828,766, the contents of which is incorporated herein in its entirety. Therefore, it should be understood that the balloon of the disclosed subject matter has at least two layers, and optionally includes one or more additional layers. The multilayer construction can include at least a first layer and a second layer having a combined wall thickness. As embodied herein, for purpose of illustration and not limitation, the first layer is made of a first polymer material having a first maximum blow-up-ratio, and the second layer is made of a second polymer material having a second maximum blow-up-ratio greater than the first maximum blow-up-ratio. The at least first and second layers define a compliance less than that of a single layer made of the first polymer material with a wall thickness equal to the combined wall thickness. Balloon in accordance with the disclosed subject matter can be formed by any suitable method.

In accordance with another aspect of the disclosed subject matter, a therapeutic agent can be disposed on the expandable member. Examples of suitable therapeutic agents include anti-proliferative, anti-inflammatory, antineoplastic, antiplatelet, anti-coagulant, anti-fibrin, antithrombotic, antimitotic, antibiotic, antiallergic and antioxidant compounds. Such therapeutic agents can be, again without limitation, a synthetic inorganic or organic compound, a protein, a peptide, a polysaccharides and other sugars, a lipid, DNA and RNA nucleic acid sequences, an antisense oligonucleotide, an antibodies, a receptor ligands, an enzyme, an adhesion peptide, a blood clot agent including streptokinase and tissue plasminogen activator, an antigen, a hormone, a growth factor, a ribozyme, and a retroviral vector.

In one embodiment, however, the therapeutic agents include a cytostatic drug. The term “cytostatic” as used herein means a drug that mitigates cell proliferation but allows cell migration. These cytostatic drugs, include for the purpose of illustration and without limitation, macrolide antibiotics, rapamycin, everolimus, zotaroliumus, biolimus, temsirolimus, deforolimus, novolimus, myolimus, structural derivatives and functional analogues of rapamycin, structural derivatives and functional analogues of everolimus, structural derivatives and functional analogues of zotarolimus and any marcrolide immunosuppressive drugs. The term “cytotoxic” as used herein means a drug used to inhibit cell growth, such as chemotherapeutic drugs. Some non-limiting examples of cytotoxic drugs include vincristine, actinomycin, cisplatin, taxanes, paclitaxel, and protaxel. Other drugs include dexamethasone, statins, sirolimus, and tacrolimus.

In addition to the therapeutic agent, any of a variety of fluid compositions can be applied to the expandable member. The fluid can include compounds or additives, such as polymers, binding agents, plasticizers, solvents, surfactants, additives, chelators, fillers, excipients, and the like, or combinations thereof. Suitable excipients, binding agents and other components include those described in detail in U.S. patent application Ser. No. 12/636,079, which is hereby incorporated by reference in its entirety. In one embodiment, excipients include poly(ethylene glycol) (PEG), polyvinylpyrrolidone (PVP), polyoxyethylene sorbitan monooleate (tweens), poloxamer triblock copolymers of poly(ethylene oxide)-poly(propylene oxide)-poly(ethylene oxide) (Pluronics), carboxymethyl cellulose (CMC), and PEG phospholipids such as 1,2-distearolyl-sn-glycero-3-phosphoethanolamine-N-(methoxy(polyethylene glycol)-2000) (PEG-PE). In one embodiment, plasticizers include PEG, propylene glycol, N-methylpyrrolidone (NMP), glycerin, and tweens. Examples of possible compounds include zotarolimus, PVP and glycerol. In one embodiment the therapeutic agent can be provided in liquid form or dissolved in a suitable solvent. In another embodiment, the therapeutic agent is provided as a particulate and mixed in a suitable carrier for application as a fluid.

The fluid compositions, such as the therapeutic agents, can be applied to the expandable member using a variety of know techniques, such as spraying (air-atomization, ultrasonic, electrostatic, piezoelectric, etc.), spray drying, pneumatic spray, spray with patterning, electrospinning, direct fluid application, dip-coating, spin-coating, pipette coating, syringe coating, vapor deposition, roll coating, micro-droplet coating, ultrasonic atomization, or other means as known to those skilled in the art. The coating can be applied over at least a length or the entirety of the expandable member. By way of example, and not limitation, certain coating processes that can be used with the instant disclosed subject matter are described in U.S. Pat. No. 6,669,980 to Hansen; U.S. Pat. No. 7,241,344 to Worsham; U.S. Publication No. 2004/0234748 to Stenzel; and U.S. Patent Application Ser. No. 61/345,575, the entire disclosures of which are hereby incorporated by reference. In accordance with one embodiment of the disclosed subject matter, the coating can be applied to either a folded or inflated balloon. Furthermore, the coating can be directly applied into the folds of the folded balloons. The coating characteristics are affected by process variables. For example, for dip-coating process, coating quality and thickness can vary as an effect of variables such as number, rate, and depth of dips along with drying time and temperature.

While the disclosed subject matter is described herein in terms of certain embodiments, those skilled in the art will recognize that various modifications and improvements can be made to the disclosed subject matter without departing from the scope thereof. Moreover, although individual features of one embodiment of the disclosed subject matter can be discussed herein or shown in the drawings of the one embodiment and not in other embodiments, it should be apparent that individual features of one embodiment can be combined with one or more features of another embodiment or features from a plurality of embodiments.

In addition to the various embodiments depicted and claimed, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

Many modifications, variations, or other equivalents to the specific embodiments described above will be apparent to those familiar with the art. It is intended that the scope of this disclosed subject matter be defined by the claims below and those modifications, variations and equivalents apparent to practitioners familiar with this art. Thus, it is intended that the disclosed subject matter include modifications and variations that are within the scope of the appended claims and their equivalents.

For example, and further to the detailed description above, the disclosed subject matter herein can include one or more of the following:

### Embodiment 1

An adjustable catheter comprising: an elongate tubular shaft having a proximal portion, a distal portion, and a length therebetween; the elongate tubular shaft having a guidewire lumen defined therein extending along at least the distal portion of elongate tubular member, the elongate tubular shaft further having at least one pressure lumen defined therein extending along at least a section of the length of the elongate tubular shaft, the at least one pressure lumen having a proximal end and a closed distal end; and a fluid adaptor in fluid communication with the at least one pressure lumen; wherein the catheter has a stiffness profile and a flexibility profile along a length thereof, at least one of the stiffness profile and the flexibility profile selectively adjustable upon introduction of a pressurizing fluid into the at least one pressure lumen.

### Embodiment 2

The catheter of Embodiment 1, wherein the at least one pressure lumen comprises a plurality of pressure lumens.

### Embodiment 3

The catheter of any of the foregoing Embodiments, wherein the plurality of pressure lumens have a uniform length

### Embodiment 4

The catheter of Embodiments 1 or 2, wherein at least one of the plurality of pressure lumens has a length different than the other pressure lumens.

### Embodiment 5

The catheter of Embodiments 1, 2, or 4, wherein each respective pressure lumen of the plurality of pressure lumens has a different length.

### Embodiment 6

The catheter of any of the foregoing Embodiments, wherein the plurality of pressure lumens are spaced circumferentially within the elongate tubular shaft.

### Embodiment 7

The catheter of any of the foregoing Embodiments, wherein the plurality of pressure lumens are in fluid communication with each other.

### Embodiment 8

The catheter of Embodiments 1 through 6, further comprising a second adaptor in fluid communication with a second of the plurality of pressure lumens to pressurize the second of the plurality of pressure lumens independently of the at least one pressure lumen.

### Embodiment 9

The catheter of Embodiments 1 through 6 and 8, further comprising a valve in communication between the adaptor and the plurality of pressure lumens to selectively pressurize at least one of the plurality of pressure lumens independent of the other pressure lumens

### Embodiment 10

The catheter of any of the foregoing Embodiments, wherein the elongate tubular shaft is a single monolithic member with the guidewire lumen and the at least one pressure lumen having the closed distal end defined therein.

### Embodiment 11

The catheter of Embodiments 1 through 9, wherein the elongate tubular shaft further includes an inner tubular member and an outer tubular member, the at least one pressure lumen being defined between the inner tubular member and the outer tubular member.

### Embodiment 12

The catheter of any of the foregoing Embodiments, wherein the elongate tubular shaft further comprises an inflation lumen extending along the length of the elongate tubular shaft.

### Embodiment 13

The catheter of any of the foregoing Embodiments, further comprising a balloon coupled to the distal portion of the elongate tubular shaft, the balloon having a proximal end, a distal end, and a working length therebetween, the balloon further having an inner chamber in fluid communication with the inflation lumen.

### Embodiment 14

The catheter Embodiments 1 through 9 or 11 through 13, wherein the guidewire lumen, the at least one pressure lumen, and the inflation lumen are in a co-axial configuration.

### Embodiment 15

The catheter of Embodiments 1 through 10 or 12 through 13, wherein the guidewire lumen, the at least one pressure lumen, and the inflation lumen extend generally parallel with each other.

### Embodiment 16

The catheter of any of the foregoing Embodiments, wherein each pressure lumen respectively includes at least one radiopaque marker proximate the closed distal end thereof.

### Embodiment 17

The catheter of any of the foregoing Embodiments, further comprising a source of pressurizing fluid to be coupled to the adaptor.

### Embodiment 18

The catheter of any of the foregoing Embodiments, further comprising a locking device to maintain a selected pressure of the pressurizing fluid in the at least one pressure lumen.

### Embodiment 19

The catheter of any of the foregoing Embodiments, further comprising a stent mounted at the distal end portion of the elongate tubular shaft.

### Embodiment 20

A method of intralumenal treatment, comprising: providing an adjustable catheter including: an elongate tubular shaft having a proximal portion, a distal portion, and a length therebetween, the elongate tubular shaft having a guidewire lumen defined therein extending along at least the distal portion of elongate tubular member, the elongate tubular shaft further having at least one pressure lumen defined therein extending along at least a section of the length of the elongate tubular shaft, the at least one pressure lumen having a proximal end and a closed distal end, and a fluid adaptor in fluid communication with the at least one pressure lumen, wherein the catheter has a stiffness profile and a flexibility profile along a length thereof, at least one of the stiffness profile and the flexibility profile selectively adjustable upon introduction of a pressurizing fluid into the at least one pressure lumen; and introducing a pressurizing fluid through the adaptor to pressurize the at least one pressure lumen to vary at least one of the stiffness profile or the flexibility profile of the adjustable catheter.

### Embodiment 21

The method of Embodiment 20, further including adjusting the pressuring fluid in the at least one pressure lumen to vary the stiffness profile or the flexibility profile of the adjustable catheter.

What is claimed is:

1. An adjustable catheter comprising:
an elongate tubular shaft having a proximal portion, a distal portion, and a length therebetween;
the elongate tubular shaft having a guidewire lumen defined therein extending along at least the distal portion of elongate tubular member,
the elongate tubular shaft further having at least one pressure lumen defined therein extending along at least a section of the length of the elongate tubular shaft, the at least one pressure lumen having a proximal end and a closed distal end; and
a fluid adaptor in fluid communication with the at least one pressure lumen;
wherein the catheter has a stiffness profile and a flexibility profile along a length thereof, at least one of the stiffness profile and the flexibility profile selectively adjustable upon introduction of a pressurizing fluid into the at least one pressure lumen.

2. The catheter according to claim 1, wherein the at least one pressure lumen comprises a plurality of pressure lumens.

3. The catheter according to claim 2, wherein the plurality of pressure lumens have a uniform length.

4. The catheter according to claim 2, wherein at least one of the plurality of pressure lumens has a length different than the other pressure lumens.

5. The catheter according to claim 4, wherein each respective pressure lumen of the plurality of pressure lumens has a different length.

6. The catheter according to claim 2, wherein the plurality of pressure lumens are spaced circumferentially within the elongate tubular shaft.

7. The catheter according to claim 2, wherein the plurality of pressure lumens are in fluid communication with each other.

8. The catheter according to claim 2, further comprising a second adaptor in fluid communication with a second of the plurality of pressure lumens to pressurize the second of the plurality of pressure lumens independently of the at least one pressure lumen.

9. The catheter according to claim 2, further comprising a valve in communication between the adaptor and the plurality of pressure lumens to selectively pressurize at least one of the plurality of pressure lumens independent of the other pressure lumens.

10. The catheter according to claim 2, wherein the elongate tubular shaft is a single monolithic member with the guidewire lumen and the at least one pressure lumen having the closed distal end defined therein.

11. The catheter according to claim 1, wherein the elongate tubular shaft further includes an inner tubular member and an outer tubular member, the at least one pressure lumen being defined between the inner tubular member and the outer tubular member.

12. The catheter according to claim 1, wherein the elongate tubular shaft further comprises an inflation lumen extending along the length of the elongate tubular shaft.

13. The catheter according to claim 12, further comprising a balloon coupled to the distal portion of the elongate tubular shaft, the balloon having a proximal end, a distal end, and a working length therebetween, the balloon further having an inner chamber in fluid communication with the inflation lumen.

14. The catheter according to claim 12, wherein the guidewire lumen, the at least one pressure lumen, and the inflation lumen are in a co-axial configuration.

15. The catheter according to claim 12, wherein the guidewire lumen, the at least one pressure lumen, and the inflation lumen extend generally parallel with each other.

16. The catheter according to claim 1, wherein each pressure lumen respectively includes at least one radiopaque marker proximate the closed distal end thereof.

17. The catheter according to claim 1, further comprising a source of pressurizing fluid to be coupled to the adaptor.

18. The catheter according to claim 1, further comprising a locking device to maintain a selected pressure of the pressurizing fluid in the at least one pressure lumen.

19. The catheter according to claim 1, further comprising a stent mounted at the distal end portion of the elongate tubular shaft.

20. A method of intralumenal treatment, comprising:
providing an adjustable catheter including:
an elongate tubular shaft having a proximal portion, a distal portion, and a length therebetween,
the elongate tubular shaft having a guidewire lumen defined therein extending along at least the distal portion of elongate tubular member,
the elongate tubular shaft further having at least one pressure lumen defined therein extending along at least a section of the length of the elongate tubular shaft, the at least one pressure lumen having a proximal end and a closed distal end, and
a fluid adaptor in fluid communication with the at least one pressure lumen,
wherein the catheter has a stiffness profile and a flexibility profile along a length thereof, at least one of the stiffness profile and the flexibility profile selectively adjustable upon introduction of a pressurizing fluid into the at least one pressure lumen; and
introducing a pressurizing fluid through the adaptor to pressurize the at least one pressure lumen to vary at least one of the stiffness profile or the flexibility profile of the adjustable catheter.

21. The method of claim 20, further including adjusting the pressuring fluid in the at least one pressure lumen to vary the stiffness profile or the flexibility profile of the adjustable catheter.
